(19) United States
(12) Patent Application Publication
(10) Pub. No.: US 2025/0195368 A1
(43) Pub. Date: Jun. 19, 2025

(54) STICK COMPOSITIONS CONTAINING BIS-ETHYLHEXYLOXYPHENOL METHOXYPHENYL TRIAZINE, LIPOPHILIC GELLING AGENT, AND A SOLVENT SYSTEM

(71) Applicant: L'OREAL, Paris (FR)

(72) Inventors: Brian Bodnar, Manasquan, NJ (US); Sang Lee Bang, Jersey City, NJ (US); Martin Josso, Chevilly-Larue (FR)

(21) Appl. No.: 18/543,489

(22) Filed: Dec. 18, 2023

(51) Int. Cl. A61K 8/49; A61K 8/02; A61K 8/73; A61K 8/92; A61Q 17/04

(52) U.S. Cl. CPC A61K 8/4966; A61K 8/0229; A61K 8/73; A61K 8/92; A61Q 17/04; A61K 2800/262; A61K 2800/49

(57) ABSTRACT

The present disclosure relates to stick compositions including 2,4-bis-{[4-(2-ethyl hexyloxy)-2-hydroxy]-phenyl}-6-(4-methoxyphenyl)-1,3,5-triazine, at least one solvent for solubilizing the bemotrizinol, and at least one lipophilic gelling agent, as well as to methods of making and using such compositions.

### FIELD OF THE INVENTION

The present disclosure relates to stick compositions comprising 2,4-bis-{[4-(2-ethyl hexyloxy)-2-hydroxy]-phenyl}-6-(4-methoxyphenyl)-1,3,5-triazine (INCI name: bis-ethylhexyloxyphenol methoxyphenyl triazine), a solvent system for solubilizing bis-ethylhexyloxyphenol methoxyphenyl triazine, and at least one lipophilic gelling agent, as well as to methods of making and using such compositions.

### DISCUSSION OF BACKGROUND

Radiation of wavelengths between 290 nm and 400 nm allows tanning of the human epidermis, while radiation of wavelengths between 290 and 320 nm, called UVB rays, hinders the development of a natural tan. Exposure is also likely to lead to a detrimental change in the biomechanical properties of the epidermis, resulting in the appearance of wrinkles leading to premature aging of the skin (i.e. photoaging).

UVA rays with wavelengths between 320 and 400 nm penetrate deeper into the skin than UVB rays. UVA rays cause immediate and persistent browning of the skin. Daily exposure to UVA rays, even for a short time, under normal conditions can damage collagen fibers and elastin, resulting in a change in the microrelief of the skin, the appearance of wrinkles and uneven pigmentation (spots, lack of uniformity of complexion).

Many studies show the need for effective protection against UVA and UVB to prevent sunburn, photoaging, and the like.

In order to obtain a high protection product, it is generally necessary to combine a large number of sunscreens and/or a high amount of UV filters to achieve high levels of filtering efficiency.

However, high levels of UV filters do not lend themselves to an easy elaboration of compositions with a stabilized and pleasant texture, particularly in the form of a stick. This is particularly true for compositions containing solid UV filter bis-ethylhexyloxyphenol methoxyphenyl triazine which is generally solubilized during formation of a stick composition, but which can be unstable as it has a tendency to recrystallize in the composition.

There remains a need in the art for improved stick compositions containing bis-ethylhexyloxyphenol methoxyphenyl triazine, particularly transparent or translucent stick compositions such as gels, which possess good protection capabilities against UV rays in addition to good application and use properties. There is also a need to inhibit bis-ethylhexyloxyphenol methoxyphenyl triazine recrystallization in stick compositions, particularly when the amount of bis-ethylhexyloxyphenol methoxyphenyl triazine present in the composition approaches or surpasses the solubility limit for the solvent(s) for solubilizing bis-ethylhexyloxyphenol methoxyphenyl triazine present in the composition.

Accordingly, one aspect of the present disclosure is a stick composition containing bis-ethylhexyloxyphenol methoxyphenyl triazine which possesses good protection properties against UV rays, good application and use properties and/or good bis-ethylhexyloxyphenol methoxyphenyl triazine recrystallization inhibition properties.

### SUMMARY OF THE INVENTION

The present disclosure relates to stick compositions comprising bis-ethylhexyloxyphenol methoxyphenyl triazine (BEMT or bemotrizinol), a solvent system for solubilizing bis-ethylhexyloxyphenol methoxyphenyl triazine, and at least one lipophilic gelling agent. Preferably, the bis-ethylhexyloxyphenol methoxyphenyl triazine is added to the compositions in an amount effective to protect keratin material against UV rays and/or is added in a super-saturated amount. Preferably, the compositions are transparent or translucent.

The present disclosure also relates to methods of making stick compositions comprising combining bis-ethylhexyloxyphenol methoxyphenyl triazine, a solvent system for solubilizing bis-ethylhexyloxyphenol methoxyphenyl triazine, and at least one lipophilic gelling agent, in the compositions during formation of the compositions. Preferably, the bis-ethylhexyloxyphenol methoxyphenyl triazine (BEMT) is added to the compositions in an amount effective to protect keratin material against UV rays and/or is added in a super-saturated amount. Preferably, the compositions are transparent or translucent.

The present disclosure also relates to methods of inhibiting bis-ethylhexyloxyphenol methoxyphenyl triazine recrystallization in a stick composition comprising bis-ethylhexyloxyphenol methoxyphenyl triazine, wherein the methods comprise adding at least one lipophilic gelling agent and/or at least one solvent system for solubilizing bis-ethylhexyloxyphenol methoxyphenyl triazine in the compositions during formation of the composition in amounts sufficient to inhibit bis-ethylhexyloxyphenol methoxyphenyl triazine recrystallization in the stick composition after formation. Preferably, the bis-ethylhexyloxyphenol methoxyphenyl triazine (BEMT) is added to the compositions in an amount effective to protect keratin material against UV rays and/or is added in a super-saturated amount. Preferably, the composition is transparent or translucent.

It is to be understood that both the foregoing general description and the following detailed description are exemplary and explanatory only, and are not restrictive of the disclosure.

### DETAILED DESCRIPTION OF THE INVENTION

In the following description and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5 includes specifically the integers within the range 1, 2, 3, 4 and 5, as well as subranges such as and 2-5, 3-5, 2-3, 2-4, 1-4, etc., as well as all fractional numbers within the range such as 1.2, 2.3, 3.4, etc., and subranges including such fractional numbers such as 1.5-3.8, 2-4.3, 4.2-4.9, etc.

“Film former”, “film-forming polymer” or “film-forming agent” as used herein means a polymer or resin which is capable of leaving a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as hydrogen atoms or chlorine atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the disclosure provided that these amounts do not materially affect at least one, preferably most, of the advantageous properties of the compositions of the disclosure. Thus, for example, “free of oil” means that an effective amount (that is, more than trace amounts) of oil(s) is/are omitted from the composition (that is, about 0% by weight), “substantially free of oil” means that oil(s) is/are present in amounts not greater than 0.1% by weight, and “devoid of oil” means that oil(s) is/are present in amounts not greater than 0.25% by weight, based on the total weight of the composition. The same nomenclature applies for all other ingredients identified throughout the application and in this paragraph such as, for example, specific UV filters and/or surfactants (compositions of the disclosure which are “free of oxybenzone and/or octinoxate,” “substantially free of oxybenzone and/or octinoxate,” and “devoid of oxybenzone and/or octinoxate,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes organic UV filters such as avobenzone, octocrylene, benzophenones, benzotriazoles and merocyanines, as well as mineral UV filters such as zinc oxide (ZnO) and titanium dioxide (TiO2).

“Anhydrous” as it is used herein means that compositions of the disclosure contain less than 3% water, meaning that the compositions can also contain less than 2% water, and less than 1% water, as well as being “free of water,” “substantially free of water,” and “devoid of water” as defined above.

“Keratinous materials” means nails (finger and/or toe nails), skin such as body, face, and eye area, scalp, keratin fibers such as eyelashes, eyebrows, and hair, and mucous membranes such as lips.

“Physiologically acceptable” means compatible with gel and having a pleasant color, odor and feel, and which does not cause any unacceptable discomfort (stinging or tautness) liable to discourage a consumer from using the composition.

“UV protection efficiency” or “filtering efficiency” in the context of the present disclosure, is evaluated from one or more of SPF, UVAPF, Critical Wavelength, and UVA-I/UV ratio.

“SPF” (Sun Protection Factor) measures the level of protection against erythema provided by a composition. The SPF value corresponds to the ratio between the minimum erythemal dosage (MED) measured wearing the composition and the MED measured with bare skin. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989).

The evaluation of the SPF (Sun Protection Factor) can be performed, for example, in vitro with spectrophotometer by Labsphere (North Sutton, NH, USA). In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the Sun Protection Factor (SPF) can also be performed in-vivo according to the ISO 24444:2019 protocol “Cosmetics-Sun protection test methods-In-vivo determination of the sun protection factor (SPF).” Also, it can be determined according to FDA protocols, as described in the document “Labeling and Effectiveness Testing; Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

The evaluation of UVA protection can also be measured in vitro with the Labsphere® spectrophotometer under conditions such as those discussed above in connection with SPF. ISO 24443:2021 protocol describes such an in vitro method. FDA broad spectrum testing procedures, in particular “critical wavelength” testing procedures, can also be found at 21 C.F.R. Part 352 Subpart D § 352.72. Also, broad spectrum testing procedures include determining the UVA1/UV ratio as described in “Sunscreen Drug Products for Over-the-Counter Human Use” published in the Federal Register https://www.federalregister.gov/documents/2019/02/26/2019-03019/sunscreen-drug-products-for-over-the-counter-human-use.” In the assay outlined in the monograph, known as the Boots adaptation of the Diffey/Robson test method, a ratio is generated of the protection afforded by the sunscreen product from UVA1 (340-400 nm) compared to the protection from total UV radiation (UVB and UVA at 290-400 nm) calculated from the absorbance curve. This ratio UVA1/UV would represent the score for the product in the in vitro test.

According to the present disclosure, compositions of the present disclosure preferably have one or more of the following properties:
Compositions have a critical wavelength as determined by FDA's critical wavelength procedures of at least 370 nm;
Compositions have an SPF value of at least 15, preferably at least 30, preferably at least 50 and preferably at least 70;
Compositions have a UVAPF/SPF ratio of at least 1/3, and preferably at least 2/5; and/or
Compositions have a UVA1/UV ratio of 0.7 or higher, preferably 0.75 or higher, and preferably 0.8 or higher.

“Makeup Result” as used herein relates to the visual impact of a composition when applied to a keratinous material such as the skin. The makeup result may relate to the apparent color of the product after application on a keratinous material or an optical effect such as shine, blurring of fine lines and imperfections, or hiding effect on pores or blemishes after the product is applied to the keratinous material.

“Long wear” as used herein refers to compositions where color or other apparent properties remains the same or substantially the same as at the time of application, as viewed by the naked eye, after an extended period of time or after a particular stress event such as water immersion or rubbing. “Long wear” may be evaluated by evaluating long wear properties by any method known in the art for evaluating such properties. For example, long wear may be evaluated by a test involving the application of a composition to keratinous material such as skin and evaluating the color of the composition after an extended period of time. For example, the color of a composition may be evaluated immediately following application to keratinous material such as skin and these characteristics may then be re-evaluated and compared after a certain amount of time. Further, these characteristics may be evaluated with respect to other compositions, such as commercially available compositions. “Long wear” may also be evaluated using vitro methods on non-keratinous substrates such as polymethylmethacrylate (PMMA), wherein properties such as color, transparency, or vitro SPF may be evaluated before and after a period of time or stressor such as water immersion, incubation at elevated temperatures, or an abrasive technique is applied.

“Super saturated amount” as used herein in the context of compositions comprising bis-ethylhexyloxyphenol methoxyphenyl triazine means an amount of bis-ethylhexyloxyphenol methoxyphenyl triazine for which at least a portion of which would be expected to be insoluble under ambient temperatures in the composition given (1) the respective amounts of bis-ethylhexyloxyphenol methoxyphenyl triazine and solvent(s) for solubilizing bis-ethylhexyloxyphenol methoxyphenyl triazine present in the composition, and (2) the solubility properties of bis-ethylhexyloxyphenol methoxyphenyl triazine in the solvent(s).

“Inhibiting bis-ethylhexyloxyphenol methoxyphenyl triazine recrystallization” as used herein means obtaining or achieving less bis-ethylhexyloxyphenol methoxyphenyl triazine recrystallization in a sample stick composition (e.g., a composition containing at least one lipophilic gelling agent such as, for example, at least one sugar fatty acid ester gelling agent) than is obtained or achieved in a comparative stick composition (e.g., a composition lacking the at least one lipophilic gelling agent).

Recrystallization of bis-ethylhexyloxyphenol methoxyphenyl triazine can be determined by incubating stick compositions comprising bis-ethylhexyloxyphenol methoxyphenyl triazine under different temperature conditions, and assessing the formula at regular timing intervals visually and/or under microscopic examination. Crystals of bis-ethylhexyloxyphenol methoxylphenyl triazine may be observed conveniently under microscopic examination using polarized light. Temperature conditions may include controlled temperatures in the range of about 4° C. or lower up to about 60° C. Temperature conditions may further include controlled cycling of the incubation temperature between ranges of about −20° C. or lower to about 20° C. or about 60° C., and holding constant the incubation temperature for a given amount of time. In some cases, the presence of bis-ethylhexyloxyphenol methoxyphenyl triazing crystals may be observed indirectly through measurements of turbidity or other spectroscopic methods (e.g. vitro SPF).

“Transparent” or “translucent” as used herein refers to compositions exhibiting a turbidity, measured according to the following method, of less than 800 NTU (Nephelometric Turbidity Units). According to preferred embodiments, turbidity is less than 500 NTU. Turbidity can be measured using a model 2100 P turbidimeter from Hach at ambient temperature (20 to 25° C.). The tubes used for the measurement are referenced AR 397 A cat 24347-06. The device is calibrated using formazine suspensions with different concentrations. Alternatively, transparency may be measured as a visual comparison between two samples held under identical treatment conditions. Further examples of measurements for transparency or translucency may involve application of the composition in a film of controlled thickness over a transparent substrate and visually inspecting the amount of light passing through the sample, or comparing the visibility of a controlled pattern placed behind the transparent substrate and controlled film of the composition under controlled lighting conditions.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means about 20-25° C.

“Atmospheric pressure” means about 760 mmHg, i.e. about 105 pascals.

“UV filter” and “sunscreen agent” are used interchangeably in this application.

“UV efficacy” and “UV efficiency” are used interchangeably in this application.

The compositions and methods of the present disclosure can comprise, consist of, or consist essentially of the essential elements and limitations of the disclosure described herein, as well as any additional or optional ingredients, components, or limitations described herein or otherwise useful. For example, the UV (ultraviolet) absorbing system of the compositions of the disclosure can “consist essentially of” bis-ethylhexyloxyphenol methoxyphenyl triazine alone, or bis-ethylhexyloxyphenol methoxyphenyl triazine in combination with oxides of zinc, titanium and/or cerium, and/or one or more organic UV filters. Also by way of example, the bis-ethylhexyloxyphenol methoxyphenyl triazine recrystallization inhibition system can “consist essentially of” sugar fatty acid ester gelling agent(s) and/or the solvent system for solubilizing bis-ethylhexyloxyphenol methoxyphenyl triazine.

For purposes of the present disclosure, the “basic and novel property” associated with compositions, components and methods which “consist essentially of” identified ingredients or actions is “inhibition of bis-ethylhexyloxyphenol methoxyphenyl triazine recrystallization.”

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Stick Composition

“Stick composition” as used herein means any composition which, is solid or semi-solid under ambient conditions. Stick compositions may be poured while hot into a mold, or shaped into a desired structure using extrusion or any other molding technique. Stick compositions may be semi-solid and held into a particular shape using other packaging means designed to dispense the semi-solid composition in a controlled manner.

According to the present disclosure, stick compositions comprising a UV (ultraviolet) absorbing system comprising 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine) are provided. “Stick composition” is understood in particular to mean a composition, the hardness of which can be measured according to the “cheese wire” method. Such compositions can contain little or no water (e.g., anhydrous, “free of,” “substantially free of,” “devoid of” water), can be in the form of an emulsion (e.g., oil-in-water or water-in-oil emulsion), etc.

The hardness of a composition may, for example, be expressed in gramforce (gf). The composition of the present disclosure may, for example, have a hardness ranging from 20 gf to 2000 gf, such as from 20 gf to 900 gf, and further such as from 20 gf to 600 gf, including all ranges and subranges therebetween.

Hardness measurement can be exemplified by the following two examples. First, the “cheese wire” method noted above, which involves cutting a 12.4 mm in diameter stick composition and measuring its hardness at 20° C. using a DFGHS 2 tensile testing machine from Indelco-Chatillon Co. or similar machine at a speed of 100 mm/minute. The hardness value from this method is expressed in grams as the shear force required to cut a stick under the above conditions. According to this method, the hardness of compositions according to the present disclosure which may be in stick form may, for example, range from 120 gf to 270 gf, preferably from 170 gf to 220 gf, for a sample of 12.4 mm in diameter stick.

If desired, another test for hardness is according to a method of penetrating a probe into the composition and in particular using a texture analyzer (for example, Texture Analyzer model PLUS-UPGRADE from Stable Micro Systems equipped with a stainless steel cylinder of height 28 mm and diameter 2 mm). The hardness measurement can be carried out at 20° C. at the center of 5 samples of the composition. The cylinder is introduced into each sample of composition at a pre-speed of 2 mm/s and then at a speed of 0.5 mm/s and finally at a post-speed of 10 mm/s, the total displacement being 2 mm. The recorded hardness value is that of the first maximum peak before the value drops observed.

The hardness of the composition of such preferred embodiments is preferably such that the compositions are self-supporting and can easily disintegrate to form a satisfactory deposit on keratinous material. In addition, this hardness may impart good impact strength to the disclosed compositions herein, which may be molded or cast, for example, in stick or dish form.

### Big-Ethylhexyloxyphenol Methoxyphenyl Triazine

According to the present disclosure, stick compositions comprising a UV (ultraviolet) absorbing system comprising 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine) are provided.

According to preferred embodiments, the UV absorbing system comprises at least 5% by weight of the bis-ethylhexyloxyphenol methoxyphenyl triazine, preferably at least 150% by weight of the bis-ethylhexyloxyphenol methoxyphenyl triazine, preferably at least 20% by weight of the bis-ethylhexyloxyphenol methoxyphenyl triazine, preferably at least 30% by weight of the bis-ethylhexyloxyphenol methoxyphenyl triazine, preferably at least 40% by weight of the bis-ethylhexyloxyphenol methoxyphenyl triazine, preferably at least 50% by weight of the bis-ethylhexyloxyphenol methoxyphenyl triazine, preferably at least 70% by weight of bis-ethylhexyloxyphenol methoxyphenyl triazine, preferably at least 80% by weight of the bis-ethylhexyloxyphenol methoxyphenyl triazine, and preferably at least 90% by weight of the bis-ethylhexyloxyphenol methoxyphenyl triazine, with all weights being based on the total weight of the UV absorbing system. “UV absorbing system” contains all organic UV filters and/or mineral UV filters present in the composition.

According to preferred embodiments, bis-ethylhexyloxyphenol methoxyphenyl triazine is present in compositions of the present disclosure in a UV absorbing effective amount such as, for example, from about 0.1% to about 10% by weight with respect to the total weight of the composition, from about 1% to about 10% by weight, from about 2.5% to about 8% by weight, and from about 4% to about 6% by weight, including all ranges and subranges therebetween such as, for example, from about 4.5% to about 10% by weight, and from about 1% to about 8% by weight, from about 1% to about 6% by weight, from about 5.5% to about 8% by weight, from about 5.7% to about 9% by weight, etc., with all weights being based on the total weight of the composition.

According to preferred embodiments, bis-ethylhexyloxyphenol methoxyphenyl triazine is present in compositions of the present disclosure in a super saturated amount.

### Lipohilic Gelling Agent

According to the present disclosure, stick compositions comprising at least one lipophilic gelling agent are provided. Preferably, the at least one lipophilic gelling agent is selected from the group consisting of sugar fatty acid ester gelling agents, semi-crystalline polymers, waxes, thixotropic agents, polyamides such as polyamide-8, polyesters such as polyester-7, amino acid based gelling agents such as gelling agents based on lysine, glutamic acid, or glutamine such as dibutyl lauroyl glutamide, styrenic block copolymers (such as those sold under the Kraton® name), and mixtures thereof. Suitable example of lipophilic gelling agents can be found, for example, in U.S. Pat. No. 10,398,631 and/or 6,921,747, the entire contents of which are hereby incorporated by reference.

### Sugar Fatty Acid Ester Gelling Agent

According to the preferred embodiments of the present disclosure, stick compositions comprising at least one sugar fatty acid ester gelling agent are provided. Any sugar-based compound esterified with at least one fatty acid can be used in accordance with the present disclosure.

Sugar-based compounds include, but are not limited to, compounds containing at least one glucose and/or fructose monomer, including sucrose. Such compounds include not only sugar monomers (e.g., glucose and fructose), but also polymeric forms of such monomers such as, for example dextrin and fructo-oligosaccharides.

Fatty acids suitable for producing an esterified compound include, but are not limited to, acid compounds containing at least C8 (8 carbon atoms) (e.g., octanoic acid). Preferably, suitable fatty acids for esterification of sugar-based compounds contain from 8 to 32 carbon atoms, preferably from 10 to 26 carbon atoms, preferably from 12 to 22 carbon atoms, and preferably from 16 to 18 carbon atoms, including saturated and unsaturated, branched and unbranched fatty acids, such as for example octanoic acid, ethylhexyl acid, stearic acid, palmitic acid, oleic acid, palmitoleic acid, etc.

Specific suitable examples of suitable fatty acid ester gelling agents for use in accordance with the present disclosure include, but are not limited to, dextrin fatty acid esters such as dextrin palmitate, dextrin stearate, and dextrin 2-ethylhexanoate/palmitate; sucrose fatty acid esters such as sucrose palmitate and sucrose stearate; and fructo-oligosaccharide fatty acid esters such as fructo-oligosaccharide stearate and fructo-oligosaccharide 2-ethylhexanoate.

According to preferred embodiments, the at least one sugar fatty acid ester gelling agent is present in compositions of the present disclosure in a bis-ethylhexyloxyphenol methoxyphenyl triazine recrystallization inhibiting effective amount such as, for example, from about 0.1% to about 30% by weight with respect to the total weight of the composition, from about 1% to about 25% by weight, from about 5% to about 20% by weight, and from about 7.5% to about 17.5% by weight, including all ranges and subranges therebetween such as, for example, from about 4.5% to about 10% by weight, and from about 12% to about 18% by weight, from about 11% to about 16% by weight, from about 5.5% to about 18% by weight, from about 7.7% to about 19% by weight, etc., with all weights being based on the total weight of the composition.

### Semi-Crystalline Polymer

According to preferred embodiments of the present disclosure, compositions comprising at least one semi-crystalline polymer are provided. “Semi-crystalline polymer” as used herein means polymer(s) comprising a crystallizable part, pendant chain or sequence in the polymer backbone (skeleton), and an amorphous part in the polymer backbone (skeleton), wherein the semi-crystalline polymer has a temperature change of reversible phase of the first order, in particular fusion (solid-liquid transition).

When the crystallizable part is a sequence of the polymeric skeleton, this crystallizable sequence is of a chemical nature different from that of the amorphous sequences; the semi-crystalline polymer is in this case preferably a block polymer, for example of the diblock, triblock or multiblock type, comprising at least one crystallizable sequence and at least one amorphous sequence. By “sequence,” it is meant that at least 5 identical repetition patterns exist. The crystallizable sequence(s) are of a chemical nature different from the amorphous sequence(s).

Preferably, the at least one semi-crystalline polymer has an average molecular mass (Mn) greater than or equal to approximately 2000 g/mol, preferably from about 2,000 to 800,000 g/mol, preferably from about 3,000 to 500,000 g/mol, preferably from about 4,000 to 150,000 g/mol, and preferably about 4,000 to 99,000 g/mol.

Apart from the crystallizable chains or sequences, the at least one semi-crystalline polymer contains chains or sequences that are amorphous. “Crystallizable chain or sequence” as used herein means a chain or sequence which, if it were alone, would pass from the amorphous state to the crystalline state, reversibly, depending on whether it is above or below the melting temperature.

“Chain” as used herein refers to a group of atoms, pendant or lateral to the backbone (skeleton) of the polymer. “Sequence” as used herein refers to a group of atoms belonging to the backbone (skeleton), and a group constituting one of the repeating units of the polymer.

According to preferred embodiments, the at least one semi-crystalline polymer contains a “crystallizable pendant chain” comprising at least 6 carbon atoms, preferably at least 8 carbon atoms, preferably at least 12 carbon atoms, and preferably at least 16 carbon atoms.

Preferably, the polymer skeleton of the at least one semi-crystalline polymer is soluble in the oily phase of the compositions of the present disclosure and introduced into the oily phase during preparation of the compositions of the present disclosure.

Preferably, the at least one semi-crystalline polymer comprises at least one alkyl acrylate chain. Preferably, the crystallizable sequences or chains of such semi-crystalline polymers containing at least one alkyl acrylate chain represent at least about 30% by weight and preferably at least about 40% by weight relative to the total weight of the semi-crystalline polymer.

Preferably, the at least one semi-crystalline polymer with crystallizable sequences are block or multi-block polymers. They can be obtained by polymerization of monomers with reactive (or ethylenic) double bonds or by polycondensation.

Preferably, when the at least one semi-crystalline polymer has crystallizable side chain(s), such polymer(s) are preferably in random or statistical form.

The at least one semi-crystalline polymer can be of synthetic origin. Preferably, the polymer(s) do not contain a polysaccharide skeleton.

The at least one semi-crystalline polymer may or may not be partially crosslinked. If crosslinked, it may involve chemical crosslinking, by reaction with a multifunctional monomer during polymerization. It can also be a physical crosslinking which can then be due either to the establishment of hydrogen or dipolar type bonds between groups carried by the polymer such as the dipolar interactions between carboxylate ionomers, these interactions being in small quantity and carried by the polymer backbone; or due to a phase separation between the crystallizable sequences and the amorphous sequences, carried by the polymer. Preferably, the at least one semi-crystalline polymer is non-crosslinked.

As indicated above, the compositions according to the present disclosure preferably comprise at least one semi-crystalline polymer containing at least one alkyl acrylate chain. Suitable semi-crystalline polymers include, but are not limited to, those resulting from the polymerization of one or more saturated alkyl (meth)acrylates. Preferably, semi-crystalline polymers with a crystallizable side chain are alkyl(meth)acrylate homopolymers, or copolymers of these monomers with a hydrophilic monomer.

According to preferred embodiments, the semi-crystalline polymer containing at least one alkyl acrylate chain comes from a monomer with a crystallizable chain chosen from saturated alkyl (meth)acrylates, preferably having a C16-C-22 chain, such as for example poly(stearyl acrylate) or poly(behenyl acrylate).

According to preferred embodiments, the semi-crystalline polymer containing at least one alkyl acrylate chain is chosen from the copolymers resulting from the polymerization of at least one monomer with a crystallizable chain chosen from the saturated alkyl (meth)acrylates, preferably having a C16-C-22 chain.

According to preferred embodiments, the semi-crystalline polymer containing at least one alkyl acrylate chain is chosen from the homopolymers obtained by polymerization of a monomer chosen from alkyl acrylates and alkyl methacrylates and among the copolymers obtained by copolymerization of a monomer chosen from alkyl acrylates and alkyl methacrylates, with a hydrophilic monomer, preferably N-vinylpyrrolidone or a hydroxyethyl (meth)acrylate, and their mixtures, and preferably a hydroxyethyl (meth)acrylate, and mixtures thereof.

Suitable examples of acceptable semi-crystalline polymers include but are not limited to homopolymers obtained by polymerization of a monomer chosen from alkyl acrylates and alkyl methacrylates, preferably having a C16-C-22 chain such as stearyl acrylates and behenyl acrylates. Specific examples include particular polymers having the name INCI Poly C10-30 Alkyl Acrylate such as the Intelimer® products from the company EVONIK such as the product Intelimer® IPA 13-1 which is a stearyl polyacrylate or the Intelimer® IPA 13-6 product which is a behenyl polymer (behenyl polyacrylate), or the Tego® SP products from the EVONIK Company.

Other suitable examples include, for example, copolymers obtained by copolymerization of a monomer chosen from alkyl acrylates and alkyl methacrylates, with a hydrophilic monomer, preferably a hydroxyethyl (meth)acrylate, such as those described in applications US 2020/0281829 and US 2020/0330334, the entire contents of which are incorporated by reference. Preferably, the copolymer is obtained by copolymerization of at least one monomer chosen from alkyl acrylates, preferably from behenyl acrylates, stearyl acrylates, and mixtures thereof, with a hydrophilic monomer such as hydroxyethyl (meth)acrylate and N-vinylpyrrolidone and preferably hydroxyethyl acrylate.

Preferably, the semi-crystalline polymer containing at least one alkyl acrylate chain is a copolymer of behenyl acrylate/2-hydroxyethyl acrylate, a copolymer of stearyl acrylate/2-hydroxyethyl acrylate and/or one of their mixtures, and in particular the polymer with the INCI name: Poly C12-22 Alkyl Acrylate/Hydroxyethylacrylate Copolymer, in particular marketed under the name Tego SP Senstar the EVONIK Company.

According to preferred embodiments, the semi-crystalline polymer(s) containing at least one alkyl acrylate chain is/are chosen from a stearyl polyacrylate, a behenyl polyacrylate, an acrylate copolymer of behenyl/2-hydroxyethyl acrylate, a copolymer of stearyl acrylate/2-hydroxyethyl acrylate, and mixtures thereof, in particular chosen from the polymer of the name INCI: Poly C10-30 Alkyl Acrylate, the copolymer of the name INCI C12-22 Alkyl Acrylate/Hydroxyethylacrylate Copolymer, and their mixtures, more particularly the polymer with the INCI name: C12-22 Alkyl Acrylate/Hydroxyethylacrylate Copolymer.

According to preferred embodiments, the at least one semi-crystalline polymer contains monomeric units of formulas (A) and (B):

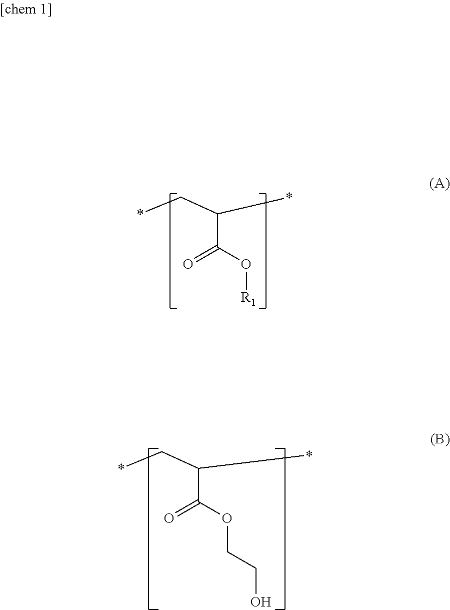

in which:
R1, independently of each other, is chosen from alkyl or alkenyl radicals, and at least 60% by weight of the R1 groups are radicals chosen from stearyl and behenyl radicals, the percentage by weight relating to the sum of all the R1 groups present in the polymer, and the weight ratio of the sum of all the hydroxyethyl acrylate units to the sum of all the acrylate units carrying the R1 group ranges from 1:30 to 1:1, and the sum of the total of units A and B is at least 95% by weight of the total weight of the polymer.

Preferably, R1 consists of alkyl radicals, preferably C16-C22 alkyl radicals, and preferably stearyl radicals (C18) or behenyl radicals (C22).

Preferably, at least 70% by weight of the R1 groups are stearyl or behenyl radicals, preferably at least 80% by weight, and preferably at least 90% by weight.

According to preferred embodiments, all the R1 groups are behenyl radicals.

According to preferred embodiments, all the R1 groups are stearyl radicals.

Preferably, the weight ratio of the sum of all the hydroxyethyl acrylate units to the sum of all the acrylate units carrying the group R1 ranges from 1:15 to 1:1, and preferably ranges from 1:10 to 1:4.

Preferably, the polymeric units present in the polymer consist of the units (A) and (B) described above.

Manufactured by Agilent Technologies, a Waters 2695 chromatographic system, a tetrahydrofuran mobile phase and detection by refractive index. The sample is filtered through a 0.45 μm nylon filter, before being injected into the liquid chromatograph. The standards used for the calibration are the Easi Vial narrow polystyrene (PS) standards from the polymer preferably has a number-average molecular weight Mn ranging from 2000 to 9000 g/mol, preferably from 5000 to 9000 g/mol. The number-average molecular weight may be measured via the gel permeation chromatography method, for example according to the method described below.

The sample is prepared by preparing a solution of the polymer at 10 mg/ml in tetrahydrofuran. The sample is placed in an oven at 54° C. for 10 minutes and then in an oscillating shaker for 60 minutes to aid dissolution. After visual inspection, the sample appeared to be totally dissolved in the solvent.

The sample prepared is analyzed using two polypore 300×7.5 mm columns Agilent Technologies. Polystyrene standards ranging from 2 520 000 to 162 daltons can be used for the calibration.

The system is equipped with a PSS SECcurity 1260 RI detector. The polystyrene calibration curve is used to determine the average molecular weight. The recording of the diagrams and the determination of the various molecular weights can be performed by the Win GPC Unichrom 81 program.

Preferably, the polymer has a melting point ranging from 40° C. to 70° C., preferably from 45° C. to 67° C. The melting point is measured by differential scanning calorimetry (DSC), for example according to methods based on standards ASTM E791 and ASTM D 34182 and the DSC calibration is performed according to standard ASTM E 9672.

According to preferred embodiments, when the polymer is such that at least 60% by weight of the R1 groups are stearyl radicals, then the polymer preferably has a melting point of from 40° C. to 60° C., preferably from 45° C. to 55° C.

According to preferred embodiments, when the polymer is such that at least 60% by weight of the R1 groups are behenyl radicals, then the polymer has a melting point of from 600° C. to 70° C., preferably from 63° C. to 67° C.

The polymer of the present disclosure can be prepared by polymerization of a monomer having the formula below:

CH₂═CH—COO—R₁,
R₁ having the meaning previously described, and of 2-hydroxyethyl acrylate.

The polymerization may be performed according to known methods, such as solution polymerization or emulsion polymerization.

The polymerization is, for example, described in document US 2007/0264204, the entire contents of which is hereby incorporated by reference.

According to preferred embodiments, the at least one semi-crystalline polymer is present in compositions of the present disclosure in an amount (active material) of from 0.05 to 10% by weight, relative to the total weight of the composition, preferably from 0.1 to 5% by weight, and preferably from 0.2 to 2% by weight, including all ranges and subranges therebetween.

### Wax

According to preferred embodiments the present disclosure, compositions comprising at least one wax are provided. Preferably, the at least one wax has a melting point of at least 40° C., preferably at least 50° C., and preferably at least 60° C.

Suitable examples of waxes that can be included in compositions of the present disclosure include those generally used in the cosmetics field such as, for example, synthetic waxes, silicone waxes, fatty alcohol waxes, waxes of natural origin, etc.

Specific waxes of natural origin include, in particular, beeswax and vegetable waxes, and include waxes such as carnauba wax, candelilla wax, ouricoury wax, Japan wax, sunflower seed wax, cork fibre wax or sugar cane wax, (rice) bran wax, montan wax, paraffin wax, lignite wax or microcrystalline wax, ceresin or ozokerite, with beeswax, carnauba wax and (rice) bran wax being preferred.

Examples of silicone waxes, if present, include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

Fatty alcohol waxes preferably contain from about 8 to about 30 carbon atoms, preferably from about 12 to about 28 carbon atoms, and preferably from about 14 to about 24 carbon atoms, including but not limited to 8 to 24 carbon atoms, 12 to 16 carbon atoms, 16 to 22 carbon atoms, etc. The fatty alcohol wax can be saturated or unsaturated, linear or branched. Mention may be made, for example, of cetyl alcohol, stearyl alcohol, behenyl alcohol, and mixtures thereof. Other examples of suitable fatty alcohol waxes include waxes including natural components (such as from one or more natural oils) and fatty alcohol components such as, for example, hydrogenated stearyl olive ester, commercially available from the supplier Sophim under the tradename Phytowax Olive 18 L 57, and hydrogenated myristyl olive ester, commercially available from the supplier Sophim under the tradename Phytowax Olive 14 L 48.

If present, the at least one wax is/are preferably present in an amount of from about 0.5% to about 30% by weight, preferably from about 1% to about 25% by weight, preferably from about 2.5% to about 25% by weight, and preferably from about 5% to about 20% by weight, including all ranges and subranges therebetween such as, for example, from about 1% to about 12% by weight, from about 2% to about 10% by weight, from about 15% to about 25% by weight, and from about 2% to about 8% by weight, with all weights being based on the total weight of the composition.

According to certain embodiments, the composition contains less than 10% by weight of beeswax, preferably less than 5% by weight of beeswax, preferably less than 2.5% by weight of beeswax, or preferably is “substantially free,” “devoid” of or “free” of beeswax as defined above.

According to certain embodiments, the composition contains less than 10% by weight of synthetic wax, preferably less than 5% by weight of synthetic wax, preferably less than 2.5% by weight of synthetic wax, or preferably is “substantially free,” “devoid” of or “free” of synthetic wax as defined above.

According to certain embodiments, the composition contains less than 10% by weight of silicone wax, preferably less than 5% by weight of silicone wax, preferably less than 2.5% by weight of silicone wax, or preferably is “substantially free,” “devoid” of or “free” of silicone wax as defined above.

### Thixotropic Agent

According to preferred embodiments, compositions of the present disclosure may include at least one thixotropic agent. The at least one thixotropic agent may be chosen, for example, from hydrophilic or organophilic clays, hydrophilic or hydrophobic fumed silicas, elastomeric organopolysiloxanes, and mixtures thereof.

Clays are silicates containing a cation that may be chosen from calcium, magnesium, aluminium, sodium, potassium, and lithium cations, and mixtures thereof. As used herein, the term “hydrophilic clay” means a clay that is capable of swelling in water; this clay swells in water and forms after hydration a colloidal dispersion.

Examples of such products include, but are not limited to, clays of the smectite family such as montmorillonites, hectorites, bentonites, beidellites, and saponites, clays of the vermiculite family, stevensite, and chlorites.

These clays may be of natural or synthetic origin.

Non-limiting examples of hydrophilic clays include smectites such as saponites, hectorites, montmorillonites, bentonites, beidellite and, in at least one embodiment, synthetic hectorites (also known as laponites), for instance, the products sold by the company Laporte under the names Laponite XLG, Laponite RD, and Laponite RDS (these products include, for example, sodium magnesium silicates and sodium lithium magnesium silicates); bentonites, for instance the product sold under the name Bentone HC by the company Rheox; magnesium aluminium silicates, which may be hydrated, for instance, the products sold by the company Vanderbilt Company under the names Veegum Ultra, Veegum HS, and Veegum DGT, and calcium silicates, such as the product in synthetic form sold by the company under the name Micro-cel C.

The organophilic clays are clays modified with chemical compounds that make the clay capable of swelling in solvent media.

The clay may be chosen, for example, from montmorillonite, bentonite, hectorite, attapulgite, sepiolite, and mixtures thereof. In one embodiment, the clay is chosen from bentonite and hectorite.

The chemical compound used to modify the organophilic clay may be chosen, for instance, from quaternary amines, tertiary amines, amine acetates, imidazolines, amine soaps, fatty sulfates, alkyl aryl sulfonates, amine oxides, and mixtures thereof.

Suitable organophilic clays include, but are not limited to, stearalkonium clays such as stearalkonium hectorite and stearalkonium bentonites such as those sold under the names Bentone 27V by the company Elementis, Tixogel LG by the company United Catalyst, and Claytone AF and Claytone APA by the company Southern Clay; quaternium-18 bentonites such as those sold under the names Bentone 3, Bentone 38, Bentone 27 V CG, and Bentone 38V by the company Elementis, Tixogel VP by the company United Catalyst, and Claytone 34, Claytone 40, and Claytone XL by the company Southern Clay; and quaternium-18/benzalkonium bentonites such as those sold under the names Claytone HT and Claytone PS by the company Southern Clay.

The hydrophilic fumed silicas may be obtained by high-temperature hydrolysis of a volatile silicon compound in an oxyhydric flame, producing a finely divided silica. Hydrophilic silicas have a large number of silanol groups at their surface. Such hydrophilic silicas are sold, for example, under the names Aerosil 1300, Aerosil 2000, Aerosil 2550, Aerosil 3000, and Aerosil 3800 by the company Degussa, and Cab-O-Sil HS-50, Cab-O-Sil EH-50, Cab-O-Sil LM-1300, Cab-O-Sil MS-550, and Cab-O-Sil M-50 by the company Cabot.

The hydrophobic fumed silicas may be obtained by modification of the surface of the silica via a chemical reaction that generates a reduction in the number of silanol groups, these groups possibly being substituted, for example, with hydrophobic groups.

The hydrophobic groups may be chosen, for instance, from:
trimethylsiloxy groups, which may be obtained by treating fumed silica in the presence of hexamethyldisilazane. Silicas thus treated are known as “Silica silylate” according to the CTFA (6th edition, 1995). They are sold, for example, under the references Aerosil R8120 by the company Degussa, and Cab-O-Sil TS-5309 by the company Cabot,
dimethylsilyloxyl or polydimethylsiloxane groups, which may be obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The at least one thixotropic agent, if present, may preferably be present in the composition in an amount from 0.05 percent by weight, for example, preferably ranging from about 0.05 percent to about 15 percent by weight, preferably from about 0.5 percent to about 10 percent by weight, and preferably from about 0.75 percent to about 5 percent by weight, all weights being relative to the total weight of the composition, including all ranges and subranges therebetween such as, for example 1% to 3%, 0.5% to 7%, 5% to 15%, etc.

### Additional Sunscreen Agents

According to preferred embodiments of the present disclosure, stick compositions optionally further comprising at least one additional UV filter (in addition to bis-ethylhexyloxyphenol methoxyphenyl triazine) selected from the group consisting of organic UV filters and mineral UV filters such as oxides of zinc, oxides of cerium, oxides of titanium, and mixtures thereof are provided.

Additional organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Suitable organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds different from Bis-Ethylhexyloxyphenol Methoxyphenyl Triazine; benzotriazole compounds; benzalmalonate compounds including those mentioned in U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from -alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. Preferably, the lipophilic organic UV filters are selected from salicylic compounds, dibenzoylmethane compounds, benzylidene camphor compounds; benzophenone compounds; triazine compounds different from Bis-Ethylhexyloxyphenol Methoxyphenyl Triazine; benzotriazole compounds; as well as other categories of compounds identified herein; and mixtures thereof.

Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β,β-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Heliopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Heliopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxybenzoyl)benzoate (such as that marketed under the tradename UVINUL A+ by BASF).

Examples of triazine compounds include Diethylhexyl butamido triazone, such as that marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzalmalonate compounds include Dineopentyl 4′-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669,323 and U.S. Pat. No. 2,463,264.

Suitable para-aminobenzoic acid compounds include PABA (p-aminobenzoic acid), ethyl PABA, Ethyl dihydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis[6-(2H-benzotriazol-2-yl)-4-methyl-phenol], such as that marketed under the trademark “Mixxim BB/200” by Fairmount Chemical, 2,2′-methylenebis[6-(2H-benzotriazol-2-yl)-4-(1,1,3,3-tetramethylbutyl)phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or “Mexoryl XL” by L'Oréal.

Examples of benzoxazole compounds include 2,4-bis[5-I(dimethylpropyl)benzoxazol-2-yl-(4-phenyl)imino]-6-(2-ethylhexyl)imino-1,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include I,I-dicarboxy(2,2′-dimethylpropyl)-4,4-diphenylbutadiene.

According to preferred embodiments, compositions of the present disclosure further comprise at least one additional organic UV filter selected from the group consisting of Avobenzone (butyl methoxydibenzoylmethane), Octisalate (ethylhexyl salicylate), Ensulizole (Phenylbenzimidazole sulfonic acid), Homosalate, Octocrylene, and mixtures thereof. In such embodiments, the UV absorbing system can “consist of” or “consist essentially of” (1) bis-ethylhexyloxyphenol methoxyphenyl triazine and (2) at least one organic UV filter selected from the group consisting of Avobenzone (butyl methoxydibenzoylmethane), Octisalate (ethylhexyl salicylate), Ensulizole (Phenylbenzimidazole sulfonic acid), Homosalate, Octocrylene, and mixtures thereof.

According to other preferred embodiments, however, compositions of the present disclosure are “free of,” “substantially free of,” or “devoid of” as defined above of one or more of additional organic UV filters selected from the group consisting of Avobenzone (butyl methoxydibenzoylmethane), Octisalate (ethylhexyl salicylate), Ensulizole (Phenylbenzimidazole sulfonic acid), Homosalate, and Octocrylene, preferably two or more, preferably three or more, preferably four or more, or preferably all five of these sunscreen agents.

According to preferred embodiments, compositions of the present disclosure are “free of,” “substantially free of,” or “devoid of” as defined above of one or more of additional organic UV filters selected from the group consisting of OXYBENZONE (benzophenone-3), OCTINOXATE (Ethylhexyl methoxycinnamate), ETHYLHEXYL TRIAZONE, DROMETRIZOLE TRISILOXANE, METHYLENE BIS-BENZOTRIAZOLYL TETRAMETHYLBUTYL PHENOL, DIETHYLAMINO HYDROXY BENZOYL HEXYL BENZOATE, DIETHYLHEXYL BUTAMIDO TRIAZONE, ISOAMYL P-METHOXYCINNAMATE, POLYSILICONE-15, 4-METHYLBENZYLIDENE CAMPHOR, DISODIUM PHENYL DIBENZIMIDAZOLE TETRASULFONATE, METHOXYPROPYLAMINO CYCLOHEXENYLIDENE ETHOXYETHYLCYANOACET ATE, preferably two or more, preferably three or more, preferably four or more, etc., and preferably “free of,” “substantially free of,” or “devoid of” all of these sunscreen agents.

According to preferred embodiments, compositions of the present disclosure are “free of,” “substantially free of,” or “devoid of” as defined above of OXYBENZONE (benzophenone-3) and/or OCTINOXATE (Ethylhexyl methoxycinnamate).

If present in compositions of the present disclosure, the at least one additional UV filter is preferably present in compositions of the present disclosure in an amount of at least about 5% by weight, preferably at least about 10% by weight, preferably at least about 12% by weight, preferably at least about 14% by weight, and preferably at least about 15% by weight, with the upper end of the range of additional UV filter present preferably being about 40% by weight (e.g., about 5-40%, about 10-40%, about 12-40%, etc.), preferably about 30% by weight (e.g., about 5-30%, about 10-30%, about 12-30%, etc.), preferably about 25% by weight (e.g., about 5-25%, about 10-25%, about 12-25%, etc.), and preferably about 20% by weight (e.g., about 5-20%, about 10-20%, about 12-20%, etc.), with all weights being based on the total weight of the composition.

According to preferred embodiments, compositions of the present disclosure comprise 10% or less by weight relative to the total weight of composition of additional UV filters, preferably less than 5% by weight relative to the total weight of composition, preferably less than 3% by weight relative to the total weight of composition, and preferably less than 1% by weight relative to the total weight of composition.

According to preferred embodiments, compositions of the present disclosure comprise 10% or less by weight relative to the total weight of composition of additional organic UV filters, preferably less than 5% by weight relative to the total weight of composition, preferably less than 3% by weight relative to the total weight of composition, and preferably less than 1% by weight relative to the total weight of composition.

According to preferred embodiments, the UV absorbing system of compositions of the present disclosure can “consist of” or “consist essentially of” bis-ethylhexyloxyphenol methoxyphenyl triazine.

According to preferred embodiments, the UV absorbing system of compositions of the present disclosure can “consist of” or “consist essentially of” (1) bis-ethylhexyloxyphenol methoxyphenyl triazine, and (2) oxides of zinc, oxides of titanium and/or oxides of cerium.

According to preferred embodiments, the UV absorbing system of compositions of the present disclosure can “consist of” or “consist essentially of” (1) bis-ethylhexyloxyphenol methoxyphenyl triazine, and (2) additional organic UV filter(s).

According to preferred embodiments, this disclosure contemplates omitting one or more of any of the specific UV filters discussed above from the UV absorbing system of compositions of the present disclosure. By way of example, octocrylene and/or octinoxate can be omitted from the compositions. Similar omission of one or more of any of the specific UV filters discussed is thus contemplated.

Addition of particular UV filters in a stick composition of the present disclosure can affect addition of further ingredients in the stick composition to achieve a suitable product. For example, addition of solid mineral UV filters can result in addition of dispersing agents such as, for example, alkali metal (e.g., potassium or sodium) silicates and polyhydroxystearic acid.

### Oil

According to preferred embodiments of the present disclosure, compositions further comprising at least one oil are provided. “Oil” means a substance which is hydrophobic and lipophilic, and is a liquid at about room temperature (20 to 25° C.) and about atmospheric pressure (760 mm Hg).

Suitable include volatile and/or non-volatile oils. Such oils can be any acceptable oil including but not limited to silicone oils and/or hydrocarbon oils.

According to certain embodiments, the compositions of the present disclosure preferably comprise one or more volatile silicone oils. Examples of such volatile silicone oils include linear or cyclic silicone oils having from 2 to 7 silicon atoms, these silicones being optionally substituted with alkyl or alkoxy groups of 1 to 10 carbon atoms. Specific oils that may be used in the disclosure include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present disclosure. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present disclosure, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

According to certain embodiments of the present disclosure, the composition comprises at least one non-volatile oil. Examples of non-volatile oils that may be used in the present disclosure include, but are not limited to, polar oils such as, for example:
esters and ethers, in particular fatty acids, such as oils of formulas R1COOR2, wherein R1 represents the remainder of a fatty acid having from 8 to 29 carbon atoms, and R2 represents a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, fatty alcohol decanoates; polyol esters, such as propylene glycol dioctanoate, neopentylglycol diheptanoate and diethylene glycol diisononanoate; tryiglycerides such as caprylic/capric acid triglycerides, for instance those sold by the company Stearineries Dubois or those sold under the names Miglyol 810, 812 and 818 by the company Dynamit Nobel; and esters of pentaerythritol such as pentaerythrityl tetraisostearate or dipentaerythrityl pentaisononanoate;
ethers containing from 10 to 40 carbon atoms;
liquid C8 to C26 fatty alcohols, for instance oleyl alcohol, cetyl alcohol, stearyl alcohol, octyldodecanol, and cetearly alcohol;
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or, for example, sunflower, corn, soybean, squash, grape seed, sesame, hazelnut, apricot, macadamia, ara, sunflower oil, castor oil, avocado, triglycerides of caprylic/capric acids such as those sold by the company Stearineries Dubois or those sold under the names Miglyol 810, 812 and 818 by the company Dynamit Nobel, coco-caprylate/caprate (esterified oil from coconut oil), jojoba oil, shea butter oil; and
mixtures thereof.

Further, examples of non-volatile oils that may be used in the present disclosure include, but are not limited to, non-polar oils such as branched and unbranched hydrocarbons, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present disclosure, the compositions of the present disclosure comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyttrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Particularly preferred oils include, but are not limited to, one or more of the following: Diisopropyl Sebacate, C12-15 Alkyl Benzoate, Phenethyl Benzoate, Isopropyl Lauroyl Sarcosinate, Diisopropyl Adipate, Dibutyl Adipate, Dicaprylyl Carbonate, Dicaprylate/Dicaprate, Coco Glycerides, Caprylic/capric triglyceride, Isopropyl Myristate, Isopropyl Palmitate, Coco Caprylate/Caprate, Ethylhexyl Palmitate, Isononyl isononanoate, Octyldodecanol, Isohexadecane, isododecane, Dicaprylyl Ether, C15-19 Alkane, diisopropyl adipate, dicaprylyl ether, and mixtures thereof.

Preferred oils for solubilizing bis-ethylhexyloxyphenol methoxyphenyl triazine, included, but are not limited to, Diisopropyl Sebacate, C12-15 Alkyl Benzoate, Phenethyl Benzoate, Isopropyl Lauroyl Sarcosinate, Dibutyl Adipate, Butyloctyl salicylate, Dicaprylyl Carbonate, Dicaprylate/Dicaprate, Coco Glycerides, Caprylic/capric triglyceride, Isopropyl Myristate, Isopropyl Palmitate, Coco Caprylate/Caprate, Ethylhexyl Palmitate, and mixtures thereof. Particularly preferred oils are C12-15 Alkyl Benzoate, Phenethyl Benzoate, Isopropyl Lauroyl Sarcosinate, Dibutyl Adipate, and Butyloctyl salicylate, with C12-15 Alkyl Benzoate and Caprylic/capric triglyceride being most preferred.

According to preferred embodiments, stick compositions of the present disclosure comprise a bis-ethylhexyloxyphenol methoxyphenyl triazine solubilizing system comprising at least one solvent which is present in the system in an amount effective to solubilize bis-ethylhexyloxyphenol methoxyphenyl triazine such that the composition is suitable for, and capable of, use as a stick composition. Preferably, the at least one solvent is selected from the oils discussed in the preceding paragraph, with C12-15 Alkyl Benzoate and Caprylic/capric triglyceride being most preferred.

According to preferred embodiments, compositions of the present disclosure contain a solvent system for solubilizing bis-ethylhexyloxyphenol methoxyphenyl triazine which “consists of” or “consists essentially of” C12-15 Alkyl Benzoate and/or Caprylic/capric triglyceride.

According to preferred embodiments, C12-15 Alkyl Benzoate and/or Caprylic/capric triglyceride are present in compositions of the present disclosure in an amount, individually or collectively, of preferably at least 10% by weight, preferably at least 20% by weight, preferably at least 30% by weight, preferably at least 40% by weight, preferably at least 50% by weight (collectively), preferably at least 60% by weight (collectively), and preferably at least 70% by weight (collectively), all weights being with respect to the total weight of the composition.

According to preferred embodiments, the at least one oil is/are present in the compositions of the present disclosure in an amount ranging from about 10% to about 80% by weight, more preferably from about 25% to about 75% by weight, and preferably from about 40% to about 70% by weight, based on the total weight of the composition, including all ranges and subranges within these ranges such as, for example, 50% to 75%, 35% to 67%, etc.

According to preferred embodiments, C12-15 Alkyl Benzoate and Caprylic/capric triglyceride are present in compositions of the present disclosure in a weight % ratio of about 5:1 to about 1:5, preferably about 3:1 to about 1:3, and preferably about 2:1 to about 1:2. Preferably, more C12-15 Alkyl Benzoate is present in the composition than Caprylic/capric triglyceride.

According to preferred embodiments, compositions of the present disclosure are anhydrous.

### Aqueous Phase

The compositions of the present disclosure may also optionally contain water. When the compositions of the present disclosure contain water, they are preferably in the form of an emulsion. Preferably, when the compositions of the present disclosure contain water, they are in the form of an emulsion containing an external aqueous phase such as an oil-in-water emulsion (O/W) or a water-in-oil-in-water emulsion (O/W/O), or an emulsion containing an external oil phase such as a water-in-oil emulsion (W/O) or an oil-in-water-in-oil (O/W/O) emulsion. Preferably, when in the form of an emulsion, the oil phase can contain silicone oils (e.g., Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

According to preferred embodiments, however, compositions of the present disclosure are devoid of water, substantially free of water, or free of water as defined herein. Preferably, the compositions of the present disclosure are anhydrous.

If present in compositions of the present disclosure, the aqueous phase may comprise at least one water-soluble organic solvent which is liquid at room temperature and atmospheric pressure. For example, such at least one water-soluble organic solvent may include:
Triethyl citrate;
C1-C5 monoalcohols having a C1-C5 alkane chain and a single hydroxyl function (OH). Suitable C1-C5 monoalcohols include methanol, ethanol, propanol, isopropanol, butanol and mixtures thereof;
polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;
and mixtures thereof.

According to preferred embodiments, the at least one water-soluble organic solvent is selected from the group consisting of ethanol, dipropylene glycol, butylene glycol, propanediol and propylene glycol, and mixtures thereof.

If present, the water-soluble organic solvent(s) is/are preferably present in compositions of the present disclosure in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

### Additional Optional Ingredients

Compositions of the present disclosure may also optionally further include at least one additive or auxiliary commonly used in cosmetic compositions and known to a person skilled in the art as being capable of being incorporated into such compositions. Such additives or auxiliaries may be chosen from film formers, coloring agents (e.g., dyes and pigments), hydrophilic gelling agents, fillers, preservatives, fragrances, surfactants, antioxidants, agents for combating free radicals, spreading agents, dispersing agents, antifoaming agents, neutralizing agents, stabilizing agents, active principles chosen from essential oils, moisturizing agents, vitamins, actives, proteins, ceramides, plant extracts, fibers, and the like, wetting agents and their mixtures. Although, preferably, compositions of the present disclosure are “free of,” “substantially free of,” or “devoid of” such additives.

### Coloring Agent

According to some embodiments, stick compositions optionally further comprising at least one coloring agent are provided. Preferably, the at least one coloring agent is chosen from the group consisting of soluble dyes, pigments, nacres and glitter.

The term “soluble dyes” should be understood to refer to organic, inorganic or organometallic compounds, soluble in the composition according to the disclosure and intended to color said composition.

Suitable dyes are, for example, Sudan Red, DC Red 17, DC Green 6, β-carotene, soybean oil, Sudan brown, DC Yellow 11, DC Violet 2, DC Orange 5 and Quinoline Yellow.

The term “nacres” should be understood to refer to iridescent particles of any shape, particularly produced by some mollusks in their shell or by synthetic means.

The term “pigments” should be understood to refer to inorganic or organic, white or colored particles of any shape, insoluble in the composition according to the disclosure and intended to color said composition.

The pigments may be white or colored, inorganic and/or organic. Of the inorganic pigments, mention may be made of titanium dioxide, optionally surface-treated, zirconium or cerium oxides, along with zinc, iron (black, yellow or red) or chromium oxides, manganese violet, ultramarine blue, chromium hydrate and iron blue, metallic powders such as aluminum powder, copper powder.

Of the organic pigments, mention may be made of carbon black, D & C type pigments, and lacquers based on cochineal carmine, barium, strontium, calcium, aluminum.

Mention may also be made of effect pigments such as particles comprising a natural or synthetic organic or inorganic substrate, for example glass, acrylic resins, polyester, polyurethane, polyethylene terephthalate, ceramics, aluminas and optionally coated with metallic substances such as aluminum, gold, copper, bronze, or with metal oxides such as titanium dioxide, iron oxide, chromium oxide, inorganic or organic pigments and mixtures thereof.

The pearlescent pigments may be chosen, for example, from pigments based on silica, titanium dioxide and mica, including for example mica-based pigments sold under the tradename Spectrval by Merck KGaA, white pearlescent pigments such as mica coated with titanium, or bismuth oxychloride, colored pearlescent pigments such as titanium mica coated with iron oxides, titanium mica coated with iron blue and chromium oxide in particular, titanium mica coated with an organic pigments of the aforementioned type and pearlescent pigments based on bismuth oxychloride.

Pigments with goniochromatic properties may be used, particularly liquid crystal or multilayer pigments.

Optical brighteners or fibers optionally coated with optical brighteners may also be used.

Preferably, the at least one coloring agent is present in the compositions of the present disclosure in an amount effective to provide visible color to gel after application of the composition to the gel, preferably ranging from about 0.1% to about 50% by weight, preferably from about 0.2% to about 40% by weight, preferably from about 0.5% to about 25%, preferably from about 5% to about 25%, and preferably from about 5% to about 20% by weight with respect to the weight of the composition, including all ranges and subranges therebetween such as, for example, 2% to 15%, 25% to 50%, 3% to 8%, etc.

### Fillers

According to some embodiments, compositions of the present disclosure may also optionally further include at least one filler.

Suitable examples of fillers include mineral or organic particles of any shape, in sheet, spherical or oblong form, regardless of the crystallographic shape (for example sheet, cubic, hexagonal, orthorhombic, etc). Mention may be made of talc, mica, kaolin, polyamide (Nylon®) (Orgasol® from Atochem), poly-β-alanine and polyethylene powders, tetrafluoroethylene polymer powders (Teflon®), lauroyl-lysine, starch, boron nitride, acrylic acid copolymers (Polytrap® from Dow Corning) and silicone resin microbeads (Tospearls® from Toshiba, for example), elastomer polyorganosiloxane particles, precipitated calcium carbonate, magnesium carbonate and hydro-carbonate, hydroxyapatite, glass or ceramic microcapsules, metallic soaps derived from carboxylic organic acids having 8 to 22 carbon atoms, preferably from 12 to 18 carbon atoms, for example zinc, magnesium or lithium stearate, zinc laurate, magnesium myristate.

The at least one filler, if present, is preferably present in an amount ranging from about 0.01% to about 15% by weight, preferably from about 0.1% to about 5% by weight, preferably from about 0.5% to about 2.5%, all weights being in relation to the total weight of the nail compositions, including all ranges and subranges therebetween.

A person skilled in the art will take care to select the optional additional additives and/or the amount thereof such that the advantageous properties of the composition according to the disclosure are not, or are not substantially, adversely affected by the envisaged addition.

Needless to say, the composition of the disclosure should be cosmetically or dermatologically acceptable, i.e., it should contain a non-toxic physiologically acceptable. The composition may be in any galenic form normally employed in the cosmetic and dermatological fields which is suitable for topical administration as discussed above.

These auxiliary additives may be present in the composition in a proportion from 0% to 99% (such as from 0.01% to 90%) relative to the total weight of the composition and further such as from 0.1% to 50% (if present), including all ranges and subranges therebetween.

According to preferred embodiments of the present disclosure, methods of protecting, enhancing the appearance of, and/or making up keratinous material by applying stick compositions of the present disclosure to the keratinous material in an amount sufficient to protect, enhance the appearance of, care for and/or makeup the keratinous material are provided.

Preferably, “making up” keratinous material includes applying a composition comprising at least one coloring agent to the keratinous material in an amount sufficient to makeup the keratinous material.

Preferably, “protecting” keratinous material includes applying a composition of the present disclosure to the keratinous material in an amount sufficient to protect the keratinous material from damage resulting from exposure to UV rays.

In accordance with the preceding embodiments, compositions of the present disclosure are applied topically to the keratinous material in an amount sufficient to protect, enhance the appearance of, care for and/or makeup the keratinous material. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

According to preferred embodiments, methods of making stick compositions comprising combining bis-ethylhexyloxyphenol methoxyphenyl triazine, a solvent system for solubilizing bis-ethylhexyloxyphenol methoxyphenyl triazine, and at least one lipophilic gelling agent, in the compositions during formation of the compositions, are provided. Preferably, the bis-ethylhexyloxyphenol methoxyphenyl triazine (BEMT) is added to the compositions in an amount effective to protect keratin material against UV rays and/or is added in a super-saturated amount. Preferably, the compositions are transparent or translucent.

According to preferred embodiments, methods of inhibiting bis-ethylhexyloxyphenol methoxyphenyl triazine recrystallization in a stick composition comprising bis-ethylhexyloxyphenol methoxyphenyl triazine, wherein the methods comprise adding at least one lipophilic gelling agent, preferably at least one sugar fatty acid ester gelling agent, and/or at least one solvent system for solubilizing bis-ethylhexyloxyphenol methoxyphenyl triazine in the composition during formation of the composition in amounts sufficient to inhibit bis-ethylhexyloxyphenol methoxyphenyl triazine recrystallization in the stick composition after formation, are provided. Preferably, the bis-ethylhexyloxyphenol methoxyphenyl triazine (BEMT) is added to the composition in an amount effective to protect keratin material against UV rays and/or is added in a super-saturated amount. Preferably, the composition is transparent or translucent.

The present disclosure also envisages kits and/or prepackaged materials suitable for consumer use containing one or more compositions according to the description herein, alone or in combination with other consumer care products such as makeup products such as basecoats, topcoats, removal compositions, etc. The packaging and application device for any subject of the disclosure may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result. The percentages are given on a weight basis.

Example 1

| | TINTED STICK |
|---|---|
| INCI US | SPF50 |
| RICINUS COMMUNIS | QS |
| (CASTOR) SEED OIL | |
| TITANIUM DIOXIDE | 0.80 |
| ISOPROPYL PALMITATE | 17.00 |
| BUTYROSPERMUM | 2.00 |
| PARKII (SHEA) BUTTER | |
| OCTINOXATE | 3.00 |
| AVOBENZONE | 3.00 |
| TOCOPHEROL | 1.00 |
| ISOHEXADECANE | 7.00 |
| THEOBROMA CACAO | 2.00 |
| (COCOA) SEED BUTTER | |
| OCTOCRYLENE | 3.50 |
| POLYETHYLENE | 14.00 |
| BEMOTRIZINOL | 5.00 |
| PARAFFIN (and) | 4.00 |
| MICROCRYSTALLINE | |
| WAX (and) SYNTHETIC | |
| WAX | |
| IRON OXIDES | 0.02 |
| IRON OXIDES | 0.01 |

Example 2

| INCI US | STICK SPF30 |
|---|---|
| OCTYLDODECANOL | 10.00 |
| RICINUS COMMUNIS | QS |
| (CASTOR) SEED OIL | |
| COPERNICIA CERIFERA | 3.00 |
| (CARNAUBA) WAX | |
| ISOPROPYL PALMITATE | 19.00 |
| EUPHORBIA CERIFERA | 6.00 |
| (CANDELILLA) WAX | |
| BUTYROSPERMUM PARKII | 2.00 |
| (SHEA) BUTTER | |
| BUTYL | 3.00 |
| METHOXYDIBENZOYLMETHANE | |
| TOCOPHEROL | 0.20 |
| POLYETHYLENE | 7.00 |
| FRAGRANCE | 0.60 |
| THEOBROMA CACAO | 2.00 |
| (COCOA) SEED BUTTER | |
| OCTISALATE | 5.00 |
| BEMOTRIZINOL | 4.00 |

Example 3

| | TRANSPARENT |
|---|---|
| INCI | STICK SPF50 |
| OCTYLDODECANOL | QS |
| HOMOSALATE | 10 |
| PROPYLHEPTYL CAPRYLATE | 10 |
| DICAPRYLYL CARBONATE | 10 |
| BEMOTRIZINOL | 5 |
| PROPYLENE GLYCOL | 8 |
| DICAPRYLATE/DICAPRATE | |
| DIISOPROPYL SEBACATE | 5 |
| C12-15 ALKYL BENZOATE | 5 |
| OCTISALATE | 5 |
| DIBUTYL LAUROYL GLUTAMIDE | 3.5 |
| DIBUTYL ETHYLHEXANOYL GLUTAMIDE | 3.5 |
| AVOBENZONE | 3 |
| DEXTRIN PALMITATE | 2 |
| TOCOPHEROL | 0.1 |

What is claimed is:

1. A stick composition comprising bemotrizinol, at least one solvent for solubilizing the bemotrizinol, and at least one lipophilic gelling agent.

2. The stick composition of claim 1, wherein the at least one lipophilic gelling agent is selected from the group consisting of sugar fatty acid ester gelling agents, semi-crystalline polymers, waxes, and mixtures thereof.

3. The stick composition of claim 1, wherein the at least one lipophilic gelling agent is selected from the group consisting of sugar fatty acid ester gelling agents, semi-crystalline polymers, and mixtures thereof.

4. The stick composition of claim 1, wherein the at least one lipophilic gelling agent is at least one wax.

5. The stick composition of claim 1, wherein the at least one lipophilic gelling agent is dextrin palmitate.

6. The stick composition of claim 1, wherein the composition is transparent.

7. The stick composition of claim 1, wherein the at least one solvent for solubilizing the bemotrizinol is selected from the group consisting of C12-15 Alkyl Benzoate, Caprylic/capric triglyceride, and mixtures thereof.

8. The stick composition of claim 1, wherein the at least one solvent for solubilizing the bemotrizinol is present in the composition in an amount ranging from about 50% to about 75% by weight with respect to the total weight of the composition.

9. The stick composition of claim 1, wherein the composition comprises 10% or less by weight relative to the total weight of composition of additional UV filters.

10. The stick composition of claim 1, further comprising at least one active agent.

11. The stick composition of claim 1, further comprising at least one coloring agent.
